(19) United States
(12) Patent Application Publication
(10) Pub. No.: US 2012/0190578 A1
(43) Pub. Date: Jul. 26, 2012

(54) Plasma Complement Components as Expression Markers for Age-Related Macular Degeneration and Related Phenotypes

(75) Inventors: Johanna M. Seddon, Boston, MA (US); John Patterson Atkinson, St. Louis, MO (US)

(73) Assignees: WASHINGTON UNIVERSITY, St. Louis, MO (US); TUFTS MEDICAL CENTER, INC., Boston, MA (US)

(21) Appl. No.: 13/388,215

(22) PCT Filed: Jul. 30, 2010
(86) PCT No.: PCT/US10/43964

§ 371 Date: Apr. 16, 2012

Related U.S. Application Data
(60) Provisional application No. 61/231,867, filed on Aug. 6, 2009.

(51) Int. Cl. C40B 30/04; C12Q 1/68; C40B 60/12; C40B 50/14; G01N 33/566; G01N 33/577

(52) U.S. Cl. 506/9; 436/501; 435/7.92; 506/39; 506/30; 435/6.11

(57) ABSTRACT

The present invention is directed to systems and method for predicting risk of AMD or a susceptibility to AMD in a patient by detecting elevated serum or plasma levels of C3, CFB or CFH and other complement factor polypeptides, wherein devated levels certain complement factors, genetic risk factors, medical risk factors, behavioral and environmental risk factors are associated with are indicative of susceptibility for or an increased risk of developing AMD, or an increased risk of progression of AMD in the patient.

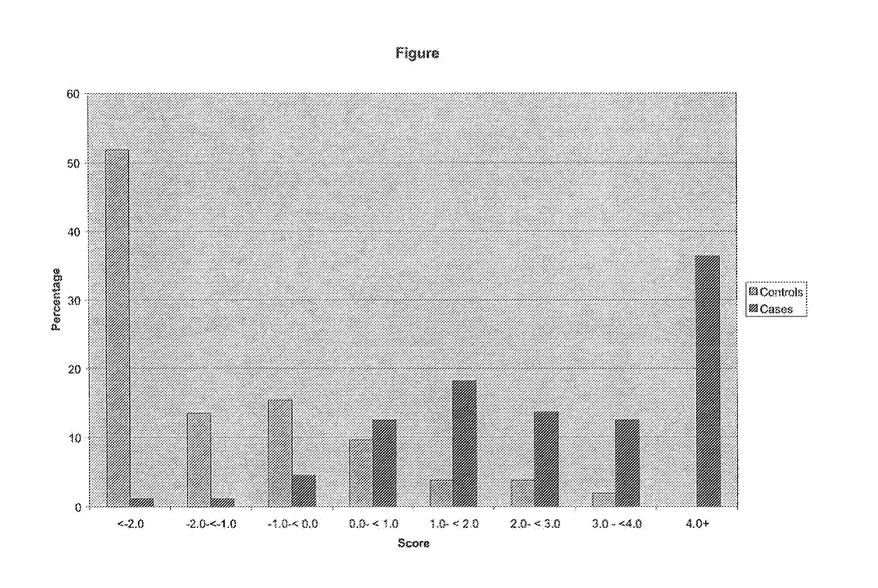

### BACKGROUND

Several genes encoding complement system components and fragments are associated with age-related macular degeneration (AMD). Alterations in circulating levels of these markers of complement activation and regulation were evaluated to determine whether they are also independently associated with advanced AMD and whether they are related to AMD genotypes.

### SUMMARY

In one aspect, the invention provides a method for determining AMD risk in a patient, comprising: obtaining a patient blood sample and determining the serum or blood plasma levels of complement factor polypeptides, wherein elevated serum or plasma levels of one or more complement factor polypeptides are indicative of susceptibility for or an increased risk of developing AMD, or an increased risk of progression of AMD in the patient.

In one embodiment the complement factor polypeptides are C3, CFB, Factor I, CFH, Factor D, Bb, C3a, iC3b, C5a, SC5b-9 and related complement pathway polypeptides. In another embodiment, elevated serum or plasma levels of complement factor polypeptides is determined using an antibody to the complement factor polypeptides. In yet another embodiment elevated serum or plasma levels of complement factor polypeptides is determined using a radial immunodiffusion assay or an ELISA, and nephelometric methods.

In another aspect the invention provides a kit for determining AMD risk in a patient, comprising: an immunoassay having antibodies directed to one or more complement factor polypeptides, reference standards comprising physiological ranges of one or more of the complement factor polypeptides, suitable packaging and instructions for use. In one embodiment the complement factor polypeptides are C3, CFB, Factor I, CFH, Factor D, Bb, C3a, iC3b, C5a, SC5b-9 and related complement pathway polypeptides.

In yet another aspect the invention provides a diagnostic system comprising: an array, the array having reference locations and diagnostic locations, the reference locations having a known quantity of an antibody to a complement factor polypeptide at each location with the known quantity of antibody differing in quantity at each location, and the diagnostic locations having a known quantity of an antibody to a complement factor polypeptide at each location with the known quantity of antibody common to each location, the diagnostic system further comprising reference standards of one or more complement factor polypeptides, an array reader, an image processor, a database having data records and information records, a processor, and an information output; wherein the system compiles and processes patient data relative to the serum or plasma levels of complement factors in a patient, and where the system outputs information relating to the statistical probability of the patient having susceptibility for or an increased risk of developing AMD, or an increased risk of progression of AMD in the patient, based on the serum or plasma levels of complement factor polypeptides in the patient. In one embodiment the complement factor polypeptides are C3, CFB, Factor I, CFH, Factor D, Bb, C3a, iC3b, C5a, SC5b-9 and related complement pathway polypeptides.

In still another aspect, the invention provides a method of using the system to predict AMD risk. The medical practitioner obtains a patient blood sample and determines the serum or plasma levels of complement factor polypeptides in the blood sample, wherein elevated serum or plasma levels of one or more complement factor polypeptides indicate a susceptibility for or an increased risk of developing AMD, or an increased risk of progression of AMD in the patient.

In one embodiment, the method includes determining the presence or absence of a particular allele at a polymorphic site associated with one or more complement pathway genes, wherein the allele indicates a susceptibility to AMD, a protective phenotype for AMD, or a neutral genotype for AMD, thereby indicating AMD risk in the patient.

In one embodiment, the allele at a polymorphic site is a single nucleotide polymorphism associated with one or more complement pathway genes including rs1061170 (Factor H gene), rs1410996 (Factor H gene), rs9332739 (Complement Factor 2 gene), rs641153 (Factor B gene), rs2230199 (C3 gene); and rs10033900 (Complement Factor I), and other genes such as rs10490924 (at LOC387715/ARM5 on chromosome 10 region). In one embodiment, the method includes the presence or absence of a particular allele is detected by a hybridization. In one embodiment, the method includes an array of genes encoding one or more complement pathway proteins. The genes include single nucleotide polymorphism associated with one or more complement pathway genes including rs1061170 (Factor H gene), rs1410996 (Factor H gene), rs9332739 (Complement Factor 2 gene), rs641153 (Factor B gene), rs2230199 (C3 gene); and rs10033900 (Complement Factor I) and other genes such as rs10490924 (at LOC387715/ARM5 on chromosome 10 region).

In one embodiment, the method includes contacting a subject sample to the diagnostic array under high stringency hybridization conditions; inputting patient information into the system; and obtaining from the system information relating to the statistical probability of the patient developing AMD.

In one embodiment, the method includes making the diagnostic array comprising: applying to a substrate at a plurality particular address on the substrate a sample of the individual purified polynucleotide compositions comprising rs1061170 (Factor H gene), rs1410996 (Factor H gene), rs9332739 (Complement Factor 2 gene), rs641153 (Factor B gene), rs2230199 (C3 gene); and rs10033900 (Complement Factor I) and other genes such as rs10490924 (at LOC387715/ARM5 on chromosome 10 region)..

In one embodiment, the method includes diagnosing AMD or a susceptibility to AMD in a subject comprising evaluating plasma levels of one or more of the complement pathway factor polypeptides of claim 2 and one or more of the gene polymorphisms of claim 11, and correlating the plasma levels of the polypeptides and the presence or absence of the gene polymorphisms, with medical, behavioral and environmental risk factors, thereby determining the patient's risk for AMD. Risk factors include hyperlipidemia, aberrant cholesterol levels, high blood pressure, obesity, smoking, vitamin and dietary supplement intake, patient use of alcohol or drugs, poor diet and sedentary lifestyle. High serum or plasma protein levels of complement factor polypeptides Bb, C3a, C5a and low serum or plasma protein levels of complement Factor H are indicative of susceptibility for or an increased risk of developing AMD, or an increased risk of progression of AMD in the patient.

In one embodiment, the predictive value of complement factors Bb and C5a are positively associated with AMD with or without the adjustments for genotype, and complement Factor H has a reverse correlation with AMD without genotype adjustments but becomes non-significant after genotype adjustments. In one embodiment, the addition of complement factors to the polymorphism-based prediction models for AMD improve the statistical significance of the correlation with AMD. Susceptibility for or an increased risk of developing AMD, or an increased risk of progression of AMD increases when the positive risk factor of complement factor C5a is integrated with the positive risk factors of genetic polymorphisms in Factor H-, Factor I- and LOC, in associated genotype prediction models.

In one embodiment, the predictive value of complement factors that are markers of chronic complement activation are significantly elevated in AMD patients compared to non-AMD patients. The complement factors that increase patient risk include Ba, C3d and Factor D.

### BRIEF DESCRIPTION OF THE DRAWING

The FIGURE is a graph showing risk scores for cases and controls based on age, gender, smoking, BMI, seven genetic variants, and C3a, Bb, and C5a fragments.

### DETAILED DESCRIPTION

The present invention provides for systems and methods for determining susceptibility for AMD, an increased risk of developing AMD, or an increased risk of progression of AMD in a patient so evaluated using the techniques and diagnostic tools described herein. Certain genetic markers indicate a risk profile for AMD. These genetic risk factors, in combination with medical risk factors, behavioral and environmental risk factors are associated with are indicative of susceptibility for or an increased risk of developing AMD, or an increased risk of progression of AMD in the patient. Elevated levels of certain complement factor polypeptides, alone or in combination with the other risk factors, provide additional details about patient risk profiles. Accordingly, serum or plasma protein levels of various complement factors are determined, and correlated with the above genetic and other risk factors, to determine a risk of AMD for a particular patient, or to monitor progression of the disease. Analysis of serum or plasma protein levels of complement factors can be determined by common techniques in the medical arts, including ELISA, radial immunodiffusion assay or nephelometric methods. Similarly, genetic determinants for AMD susceptibility can be identified through common techniques such as PCR and nucleic acid sequencing.

In one aspect, the invention comprises an array of polypeptide or polynucleotides, particularly including those SNPs given below and probes for detecting the allele at the SNPs. Polynucleotide arrays provide a high throughput technique that can assay a large number of polynucleotide sequences in a single sample. This technology can be used, for example, as a diagnostic tool to assess the risk potential of developing AMD using the SNPs and probes of the invention. Polynucleotide arrays (for example, DNA or RNA arrays), include regions of usually different sequence polynucleotides arranged in a predetermined configuration on a substrate, at defined x and y coordinates. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Arrays can be fabricated by depositing previously obtained biopolymers onto a substrate, or by in situ synthesis methods. The substrate can be any supporting material to which polynucleotide probes can be attached, including but not limited to glass, nitrocellulose, silicon, and nylon. Polynucleotides can be bound to the substrate by either covalent bonds or by non-specific interactions, such as hydrophobic interactions. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266; U.S. Pat. No. 6,232,072; U.S. Pat. No. 6,180,351; U.S. Pat. No. 6,171,797; EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Other techniques for fabricating biopolymer arrays include known light directed synthesis techniques. Commercially available polynucleotide arrays, such as Affymetrix GeneChip™, can also be used. Use of the GeneChip™, to detect gene expression is described, for example, in Lockhart et al., Nat. Biotechnol., 14:1675, 1996; Chee et al., Science, 274:610, 1996; Hacia et al., Nat. Gen., 14:441, 1996; and Kozal et al., Nat. Med., 2:753, 1996. Other types of polypeptide and polynucleotide arrays are known in the art, and are sufficient for developing an AMD diagnostic array of the present invention, for example an ELISAspot assay. To create the arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences of the SNPs provided herein, or the complement thereof. Preferred arrays comprise at least one single-stranded polynucleotide probe comprising at least 6, 7, 8, 9, 10, 11, 12, 13, 14, 15, 16, 17, 18, 19, 20, 25, or 30 or more contiguous nucleotides selected from the nucleotide sequences or the complement thereof

Tissue samples from a subject can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels that can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance. Preferably, after the array has been exposed to a sample, the array is read with a reading apparatus (such as an array “scanner”) that detects the signals (such as a fluorescence pattern) from the array features. Such a reader preferably would have a very fine resolution (for example, in the range of five to twenty microns) for a array having closely spaced features.

The signal image resulting from reading the array can then be digitally processed to evaluate which regions (pixels) of read data belong to a given feature as well as to calculate the total signal strength associated with each of the features. The foregoing steps, separately or collectively, are referred to as “feature extraction” (U.S. Pat. No. 7,206,438). Using any of the feature extraction techniques so described, detection of hybridization of a patient derived polynucleotide sample with one of the AMD markers on the array given as SEQ ID NO:1-7 identifies that subject as having or not having a genetic risk factor for AMD, as described above.

In another aspect, the invention provides a system for compiling and processing patient data, and presenting a risk profile for developing AMD. A computer aided medical data exchange system is preferred. The system is designed to provide high-quality medical care to a patient by facilitating the management of data available to care providers. The care providers will typically include physicians, surgeons, nurses, clinicians, various specialists, and so forth. It should be noted, however, that while general reference is made to a clinician in the present context, the care providers may also include clerical staff, insurance companies, teachers and students, and so forth. The system provides an interface, which allows the clinicians to exchange data with a data processing system. The data processing system is linked to an integrated knowledge base and a database. The database may be software-based, and includes data access tools for drawing information from the various resources as described below, or coordinating or translating the access of such information. In general, the database will unify raw data into a useable form. Any suitable form may be employed, and multiple forms may be employed, where desired, including hypertext markup language (HTML) extended markup language (XML), Digital Imaging and Communications in Medicine (DICOM), Health Level Seven.™. (HL7), and so forth. In the present context, the integrated knowledge base is considered to include any and all types of available medical data that can be processed by the data processing system and made available to the clinicians for providing the desired medical care. In general, data within the resources and knowledge base are digitized and stored to make the data available for extraction and analysis by the database and the data processing system. Even where more conventional data gathering resources are employed, the data is placed in a form that permits it to be identified and manipulated in the various types of analyses performed by the data processing system.

The integrated knowledge base is intended to include one or more repositories of medical-related data in a broad sense, as well as interfaces and translators between the repositories, and processing capabilities for carrying out desired operations on the data, including analysis, diagnosis, reporting, display and other functions. The data itself may relate to patient-specific characteristics as well as to non-patient specific information, as for classes of persons, machines, systems and so forth. Moreover, the repositories may include devoted systems for storing the data, or memory devices that are part of disparate systems, such as imaging systems. As noted above, the repositories and processing resources making up the integrated knowledge base may be expandable and may be physically resident at any number of locations, typically linked by dedicated or open network links. Furthermore, the data contained in the integrated knowledge base may include both clinical data (e.g., data relating specifically to a patient condition) and non-clinical data. Examples of preferred clinical data include patient medical histories, patient serum and cellular antioxidant levels, and the identification of past or current environmental, lifestyle and other factors that predispose a patient to develop AMD. These include but are not limited to various risk factors such as obesity, smoking, vitamin and dietary supplement intake, use of alcohol or drugs, poor diet and a sedentary lifestyle. Non- clinical data may include more general information about the patient, such as residential address, data relating to an insurance carrier, and names and addresses or phone numbers of significant or recent practitioners who have seen or cared for the patient, including primary care physicians, specialists, and so forth.

The flow of information can include a wide range of types and vehicles for information exchange. In general, the patient can interface with clinicians through conventional clinical visits, as well as remotely by telephone, electronic mail, fauns, and so forth. The patient can also interact with elements of the resources via a range of patient data acquisition interfaces that can include conventional patient history fauns, interfaces for imaging systems, systems for collecting and analyzing tissue samples, body fluids, and so forth. Interaction between the clinicians and the interface can take any suitable form, depending upon the nature of the interface. Thus, the clinicians can interact with the data processing system through conventional input devices such as keyboards, computer mice, touch screens, portable or remote input and reporting devices. The links between the interface, data processing system, the knowledge base, the database and the resources typically include computer data exchange interconnections, network connections, local area networks, wide area networks, dedicated networks, virtual private network, and so forth.

In general, the resources can be patient-specific or patient-related, that is, collected from direct access either physically or remotely (e.g., via computer link) from a patient. The resource data can also be population-specific so as to permit analysis of specific patient risks and conditions based upon comparisons to known population characteristics. It should be noted that the resources can generally be thought of as processes for generating data. While many of the systems and resources will themselves contain data, these resources are controllable and can be prescribed to the extent that they can be used to generate data as needed for appropriate treatment of the patient. Exemplary controllable and prescribable resources include, for example, a variety of data collection systems designed to detect physiological parameters of patients based upon sensed signals. Such electrical resources can include, for example, electroencephalography resources (EEG), electrocardiography resources (ECG), electromyography resources (EMG), electrical impedance tomography resources (EIT), nerve conduction test resources, electronystagmography resources (ENG), and combinations of such resources. Various imaging resources can be controlled and prescribed as indicated at reference numeral. A number of modalities of such resources are currently available, such as, for example, X-ray imaging systems, magnetic resonance (MR) imaging systems, computed tomography (CT) imaging systems, positron emission tomography (PET) systems, fluorography systems, sonography systems, infrared imaging systems, nuclear imaging systems, thermoacoustic systems, and so forth. Imaging systems can draw information from other imaging systems, electrical resources can interface with imaging systems for direct exchange of information (such as for timing or coordination of image data generation, and so forth).

In addition to such electrical and highly automated systems, various resources of a clinical and laboratory nature can be accessible. Such resources may include blood, urine, saliva and other fluid analysis resources, including gastrointestinal, reproductive, and cerebrospinal fluid analysis system. Such resources can further include polymerase (PCR) chain reaction analysis systems, genetic marker analysis systems, radioimmunoassay systems, chromatography and similar chemical analysis systems, receptor assay systems and combinations of such systems. Histologic resources, somewhat similarly, can be included, such as tissue analysis systems, cytology and tissue typing systems and so forth. Other histologic resources can include immunocytochemistry and histopathological analysis systems. Similarly, electron and other microscopy systems, in situ hybridization systems, and so forth can constitute the exemplary histologic resources. Pharmacokinetic resources can include such systems as therapeutic drug monitoring systems, receptor characterization and measurement systems, and so forth. Again, while such data exchange can be thought of passing through the data processing system, direct exchange between the various resources can also be implemented.

Use of the present system involves a clinician obtaining a patient sample, and evaluation of the presence and amount of a complement factor protein or genetic marker in that patient indicating a predisposition (or not) for AMD, alone or in combination with other known risk factors. The clinician or their assistant also obtains appropriate clinical and non-clinical patient information, and inputs it into the system. The system then compiles and processes the data, and provides output information that includes a risk profile for the patient, of developing AMD.

The present invention thus provides for certain markers that have been correlated to AMD. These markers are useful as diagnostics, and are preferably used to fabricate an array, useful for screening patient samples. The array, in a currently most preferred embodiment, is used as part of a laboratory information management system, to store and process additional patient information in addition to the patient's genomic profile. As described herein, the system provides an assessment of the patient's risk for developing AMD, risk for disease progression, and likelihood of disease prevention based on patient controllable factors.

Plasma and DNA samples were selected from individuals in a previously described AMD registry who progressed to the advanced stages of AMD, including 58 with geographic atrophy and 62 with neovascular disease. Subjects without AMD of similar age and gender were included as controls (n=60). Plasma complement components (C3, CFB, CFI, CFH, Factor D) and activation fragments (Bb, C3a, C5a, iC3b, SC5b9) were analyzed. DNA samples were genotyped for seven single nucleotide polymorphisms in six genes previously shown to be associated with AMD: CFB, CFH, C2, C3, CFI, and the LOC387715/ARMS2 gene region. Association between AMD and each complement biomarker was assessed using logistic regression, controlling for age, gender, and pro-inflammatory risk factors: smoking and body mass index. Functional genomic analyses were performed to assess the relationship between the complement markers and genotypes. Concordance or “C” statistics were calculated to assess the effect of complement components and activation fragments on our AMD gene-environment prediction model.

The highest quartiles of Bb and C5a were discovered to be significantly associated with advanced AMD compared with the lowest quartiles. In multivariate models without genetic variants, the odds ratio (OR) for Bb was 3.3 (95% confidence interval (CI) 1.3-8.6) and the OR for C5a was 3.6 (95% CI 1.2-10.3). Controlling for genetic variants, these ORs were substantially higher. The alternative pathway regulator CFH was inversely associated with AMD in the model without genotypes (OR 0.3, p=0.01). Positive associations were found between BMI and plasma C3, CFB, CFH, iC3b and C3a. There were also significant associations between C5a fragment and LOC387715/ARMS2 and C3 genotypes (p for trend=0.02, 0.04), respectively. C statistics for models with behavioral and genetic factors increased to 0.94±0.20 with the addition of C3a, Bb and C5a.

Increased levels of activation fragments Bb and C5a are independently associated with AMD. Higher BMI is related to increased levels of complement components. C5a is associated with AMD genotypes. C statistics are stronger with the addition of C3a, Bb and C5a in predictive models. Results implicate ongoing activation of the alternative complement pathway in AMD pathogenesis.

In our analyses we found an independent relationship between activation fragments Bb and C5a and advanced AMD. Our study provides new information regarding the association between the complement system and AMD in several ways. We evaluated each complement component or fragment both with and without seven AMD genotypes, controlling for other covariates including age, gender, smoking, and BMI. We found a significant association between median levels of fragments Bb and C5a and advanced AMD as well as the GA group separately. There was a significant inverse association between CFH component and the total AMD group and GA group separately. C3a levels were significantly higher among cases but this association did not persist after controlling for other genetic and non-genetic factors.

Associations were found between higher BMI and activation fragments C3a, iC3b, and component CFH among controls, as well as associations between BMI and components C3, CFB, and CFH among the entire study population, while controlling for AMD. Higher BMI is a risk factor for developing AMD, has been shown to be related to higher CRP,1 and the independent association between BMI and complement components and fragments as disclosed herein are also noteworthy. A significant inverse association was found between C5a fragment and LOC387715/ARMS2 genotypes, and significant positive trend between C3 genotypes and higher C5a fragment. ROC curves and C statistics were calculated for C3a, Bb, and C5a which included demographic and environmental covariates and seven SNPs. Significant increases in C statistics were observed when these fragments were added to the prediction model, especially when they were considered together, with discrimination between advanced AMD and a control increasing to as high as 94%.

For AMD there is increased complement deposition in Bruch's membrane and in drusen. Photo-oxidation of bis-retinoid lipofuscin in cultured RPE cells has been shown to lead to complement activation and release of fragments iC3b and C3a in vitro. This finding supports the theory that increased complement activation AMD might occur as a result of photo-oxidation within RPE. Having insufficient functional CFH to dampen the complement-induced injury in the outer retina may then lead to pathologic features in advanced AMD. This line of thinking also is supported by the inverse relationship found between CFH and AMD for analyses that were not controlled by genotype. The major predisposing effect is thus one of decreased regulation of activation of the complement cascade in the retina, such as through dysfunctional CFH gene protein or from insufficient production of protein. Whether there is systemic activation of the complement system or the elevated levels reflect systemically circulating fragments from local activation in the eye (as is observed in rheumatoid arthritis with complement activation in a joint) is not clear. These are not mutually exclusive possibilities in that both systemic activation and local (e.g., retinal) complement activation could play a role. The latter is established based on findings of complement deposition in drusen. Biologically the same outcome would occur if CFH was present in lower than normal amounts or was not fully functional. Complement activation, while probably not the initiating cause of the injury in AMD, can nevertheless substantially contribute to subsequent tissue damage. Animal models of laser induced damage to Bruch's membrane provide evidence for the importance of C5a and other components of alternative pathway in the development of choroidal neovascularization. Elevated plasma activation fragments Bb, C3a, and C5a are consistent with continuous, low level alternative pathway activation in patients with advanced AMD. Subtle alterations in the efficiency of activation and/or in regulatory capacity negatively influence a pathologic process that plays out over the years.

Activation of the complement system can lead to the formation the membrane attack complex (MAC or C5b-9). This terminal pathway begins with the cleavage of C5 to C5a and C5b. SC5b-9 is the fluid phase terminal complex which usually circulates bound to the inhibitor vitronectin. The quantity of SC5b-9 in the circulation is in part a reflection of the MAC that is generated locally. Thus, a fraction will deposit at the site and part will be in the fluid phase. Described herein are data showing increased activation of C5.

C5a levels in the blood and deposition of C5b-9 in the retina in AMD patients. C5a can bind to its receptor, C5aR, and has been shown to be important in models of AMD. C5b can be generated by activation of the alternative pathway or by direct cleavage by proteases including trypsin and thrombin. A recent report provided considerable evidence to indicate C5 activation by thrombin in a lung model of immune complex activation. As described herein, the SC5b-9 is not increased in AMD.

The association described herein between high BMI and elevated C3, CFB and CFH as well as increased concentrations of complement activation fragments derived from several components of the alternative pathway point to a role of the complement system in obesity. Adipose tissue is the source of Acylation Stimulating Protein (ASP), which is also C3a desArg. C3a desArg is a C3 derived protein formed by removal of an arginine from the carboxy-terminus of C3a and is the major form of circulating C3a. Before the relationship to C3 was discovered, a role for lipogenic function of ASP/C3a desArg was discovered. Sivaprasad et al. studied C3a desArg as an indicator of C3 systemic complement activation among 84 cases of advanced AMD (42 GA, 42 NV), as well as in association with the CFHrs1061170 genotype. They found no significant association between genotype and C3a desArg, but did find an increase in C3a desArg concentration among cases. Described herein are associations with C3a and AMD, as well as a second C3 split product, iC3b with BMI. Further, adipsin, an adipose specific factor linked to adipocyte differentiation, was shown to be Factor D of the alternative pathway of the complement system. No association was found with Factor D. Other noteworthy adipose-complement alterations include altered lipid clearance in C3^{-/-} mice and partial lipodystrophy in children with an autoantibody that stabilizes the alternative pathway C3 convertase. Also, there are a few reports of elevated C3 concentrations in individuals with a high BMI. The data described herein support increased turnover and activation of the alternative pathway in individuals with high BMI, evidenced by increased activation fragments, Bb, C3a and iC3b. Enhanced alternative pathway activation in obesity and in AMD may cooperate to accelerate tissue damage.

Another noteworthy finding was the OR for the relationship between AMD and LOC387715/ARMS2 increased with the addition of C5a into the model. One possibility is that a tissue metalloproteinase directly activates C5 leading to formation of C5a and C5b, with former binding to its receptor and the latter beginning the MAC.

Regardless of known genotype, both Bb and C5a are strongly associated with increased risk of advanced AMD. Described herein are a new association between AMD and Bb and a new significant inverse association with median level of CFH. The case population consisted solely of advanced AMD cases, the majority of which were documented progressors to GA and NV disease. The GA and NV groups are in similar proportion and results are displayed for each subtype. The relationships between AMD and complement components and fragments has thereby been expanded by considering six genes (seven genetic loci) and covariates known to be related to AMD. The effect of these genetic factors was determined on function or levels of the complement factors. Data included herein also evidences increased BMI in association with complement activation fragments, suggesting that BMI itself increases the chronic activation of the complement system. The AMD prediction model with genetic, demographic, and environmental factors was improved with the addition of plasma complement markers. Results provide new evidence that the complement system, and particularly the alternative pathway, is chronically activated in AMD.

### EXEMPLIFICATION

Several genes in the complement pathway are associated with age related macular degeneration (AMD) including: CFH, CFB, C2, C3, and CFI. Another gene in the LOC387715/ARMS2 region on chromosome 10 is related to AMD, although the mechanism is uncertain. Behavioral and modifiable factors, such as smoking and body mass index (BMI) that are related to AMD, influence levels of inflammatory biomarkers and also modify genetic susceptibility. The levels of complement components (C3, CFB, CFI, CFH, factor D) and activation fragments (Bb, C3a, C5a, iC3b, SC5b-9) in plasma samples from cases with advanced AMD and controls, were tested to further assess the role of the complement system and its association with AMD. The association of these biomarkers with behavioral factors related to AMD and their association with AMD genotypes were also assessed. The degree to which these components and activation fragments contribute to risk to predict the prevalence and incidence of advanced AMD was therefore identified.

Study Population

Patients with and without AMD were enrolled in genetic epidemiologic studies using standardized clinical examinations, questionnaires and fundus photography. Grades were based on clinical and fundus photographic data using the Clinical Age-Related Maculopathy Grading System (CARMS). From the AMD registry, 180 unrelated Caucasian individuals with DNA and plasma samples were selected. The 120 cases (60 male and 60 female), were composed of 58 individuals with geographic atrophy (GA), and 62 with neovascular disease (NV). Among the cases, 108 had a baseline CARMS grade of 1, 2, or 3 and progressed to either grade 4-central or non-central GA (n=48), or grade 5 with NV (n=62) in one or both eyes. Controls (n=60) had a CARMS grade of 1 in both eyes, and consisted of 30 males and 30 females. The mean±standard deviations (SD) of ages of the case and control groups were 82±6.9 years and 79±4.4 years, respectively. This research followed the tenets of the Declaration of Helsinki, was approved by the institutional review board, and informed consent was obtained from all subjects.

Genotyping

DNA samples were genotyped for seven single nucleotide polymorphisms (SNPs) related to AMD: 1) Complement Factor H (CFH)Y402H (rs1061170) in exon 9 of the CFH gene on chromosome 1q32, a change 1277T>C, resulting in a substitution of histidine for tyrosine at codon 402 of the CFH protein, 2) CFH rs1410996 an independently associated SNP variant within intron 14 of CFH, 3) LOC387715 A69S ARMS2 (rs10490924 in the LOC387715/ARMS2 region of chromosome 10, a non-synonymous coding SNP variant in exon 1 of LOC387715), resulting in a substitution of the amino acid serine for alanine at codon 69, 4) Complement Factor 2 or C2 E318D (rs9332739), the non-synonymous coding SNP variant in exon 7 of C2 resulting in a substitution of aspartic acid for glutamic acid at codon 318, 5) Complement Factor B or CFB R32Q (rs641153), the non-synonymous coding SNP variant in exon 2 of CFB resulting in the substitution of the amino acid glutamine for arginine at codon 32, 6) Complement Factor 3 or C3 R102G (rs2230199), the non-synonymous coding SNP variant in exon 3 of C3 resulting in the substitution of the amino acid glycine for arginine at codon 102, 7) Complement Factor I or CFI (rs10033900), an independently associated SNP located in the linkage peak region of chromosome 4, 2781 base pairs upstream of the 3′ untranslated region of CFI.

The HTRA1 gene, adjacent to the genetic variant on chromosome 10, LOC387715A69S, may in fact be the AMD-susceptibility gene on 10q26 as the relevant SNPs in these two genes have been reported to be nearly perfectly correlated. Thus, while the other SNP is a promising candidate variant, rs10490924 used in this study can be considered a surrogate for the causal variant that resides in this region. For the C2/CFB genes, there are two independent associations to the C2/CFB locus. Genotyping was performed using primer mass extension and MALDI-TOF MS analysis (MassEXTEND methodology of Sequenom, San Diego, Calif.).

Plasma Samples

Fasting plasma samples were drawn into Becton Dickinson tubes containing K₂ EDTA, primarily at time of baseline grade. The blood was centrifuged and plasma separated within 30 minutes of collection. Samples were frozen and stored in liquid nitrogen until testing was performed. The following complement activation fragments and complement system proteins were analyzed: Bb, C3a, C5a, iC3b, SC5b-9, C3, Factor D, CFB, CFH, and CFI.

Complement Components and Activation Fragments

C3 was measured by standard clinical laboratory nephelometric methods. CFB, CFH and CFI were measured by radial immunodiffusion (RID) using goat anti-human antibodies specific for the individual proteins in 1% agarose gels. The CFH antiserum (Quidel, San Diego Calif.) was produced against purified human factor H. It is specific for CFH but has not been evaluated for cross-reactivity to the CFH-related proteins. The complement laboratory reference range for CFH is 160-412 μg/mL, in agreement with ranges used by other complement reference laboratories in the United States and Europe. The antibodies used for CFB and CFI are also goat polyclonal antisera specific for the given proteins (Quidel, San Diego Calif.). Purified CFB, CFH and CFI were used to standardize the assays (Quidel, San Diego Calif. and CompTech, Tyler Tex.). Appropriate controls were included with each batch of patient samples, and the results calculated from the area under the precipitin rings.

Factor D was measured with a Quantikine® ELISA kit (R&D Systems, Minneapolis, Minn.) that uses a plate precoated with specific monoclonal antibody for Factor D. The standard provided with the kit is 40 ng of recombinant human complement factor D (CFD). The enzyme conjugate is polyclonal anti-CFD linked to horse-radish peroxidase. Three controls: high, medium and low are run with each set of samples. The reference range given by the manufacturer is 1468-3657 ng/mL for EDTA plasma. The reference range established by the complement laboratory is 1688-3076 ng/mL for EDTA plasma, which was converted to μg/mL: 1.69-3.08 (mean±2 SD) for reporting in this study.

Complement activation markers were measured by ELISAs using commercially available kits with extra in-house controls. Bb, iC3b and SC5b-9 were done with kits from Quidel (San Diego, Calif.). The tests for C3a and C5a were performed using OptEIA® kits from BD-Pharmingen (San Diego, Calif.). Reference ranges for all of the complement fragments were established in the complement lab under stringent validation protocols. Reference ranges for the assays are as follows: C3a (305-1239 ng/mL), C5a (5.0-25.4 ng/mL), Bb (0.41-1.49 μg/mL), iC3b (0-17.4 ng/mL), and SC5b-9 (72-244 ng/mL). The complement laboratory is accredited by the College of American Pathologists and the Clinical Laboratory of 1988 to perform tests of high complexity.

Covariates

Data on smoking were collected from a standardized risk factor questionnaire. Smokers were defined as having smoked at least one cigarette per day for six months or longer. Pack years were calculated by multiplying number of cigarettes smoked per day by number of years smoked divided by twenty. Height and weight were measured at the time of the baseline grade to calculate BMI (weight in pounds multiplied by 703 divided by height in inches squared), or in a few instances by self-report.

Statistical Analysis

Odds ratios (ORs) and 95% confidence intervals (CIs) were calculated for covariates, and genotypes using logistic regression (controlling for age (60-79, 80+and gender) to evaluate their association with each maculopathy group (GA and NV), and total AMD (GA and NV combined), with controls. T-tests were used to calculate p values for age between cases and controls. P values ≦0.05 were considered statistically significant for all analyses.

The Wilcoxon Rank Sum test was used to calculate p values to assess the relationship between the median plasma level of complement components and activation fragments and maculopathy group.

ORs and 95% CIs for total AMD were computed to compare the 4th quartile to the 1st quartile of component and activation fragment using logistic regression. In Model A, controls for age (60-79, 80+), gender, BMI (<25, 25-29.9, 30+) and smoking (ever, never) were considered. In Model B, controls for the same factors as in Model A were considered, plus all of the genotypes: CFB (CC, CT/TT), CFHY402H (TT, CT, CC), CFH:rs1410996 (TT, CT, CC), C2 (GG, CG/CC), LOC387715/ARMS2 (GG, GT/TT), C3 (CC, CG, GG), and CFI (CC, CT, TT).

ORs and 95% CIs for AMD were calculated for each genotype separately with one component or activation fragment at a time to assess whether the effect of genotype was mediated by that complement component or activation fragment. Log component and activation fragment values were used, because the distribution was slightly skewed, to assess associations with genotype among controls using linear regression. In addition, linear regression was done to test the relationship between each complement component or activation fragment and smoking and BMI, both a) among controls and b) among cases and controls combined. To see if the effects of genotype and complement components and activation fragments were dependent on one another, logistic regression was used to test for interaction effects on risk of AMD.

General linear model analysis was used to calculate the least square means to assess the relationship between mean level of components or fragments and genotype among cases and controls combined. In this model, controls for age, gender, AMD status and all the genotypes were used.

C Statistics were calculated to assess if activation fragments contribute to the predictability of developing advanced AMD. The area under the receiver operating characteristic (ROC) curve was obtained, and an age-adjusted concordant or “C” statistic based on the ROC curve was calculated. C statistics was calculated for six models with varying combinations of covariates, genotypes, and activation fragments to assess the probability that the risk score from a random case was higher than the corresponding risk score from a random control, based on the group of risk factors in each model, such that a perfect score would be 1.0, or 100% predictability. We obtained standard errors of estimated C statistics and compared C statistics from alternative risk prediction models using correlated ROC curve methods.

TABLE 1
Baseline Demographic, Behavioral, and Genetic Factors According to Maculopathy Group

| | Maculopathy Group | | | | | | | | |
|---|---|---|---|---|---|---|---|---|---|
| Controls | GA | | | NV | | | Total AMD | | |
| N (%) | N (%) | | | N (%) | | | N (%) | | |
| (n = 60) | (n = 58) | OR (95% CI)* | p value | (n = 62) | OR (95% CI)* | p value | (n = 120) | OR (95% CI)* | p value |

Characteristics

| | | | | | | | | | | |
|---|---|---|---|---|---|---|---|---|---|---|
| Mean (SD) | 79 (4.4) | 82 (7.9) | | 0.07^{†} | 82 (5.7) | | 0.003^{†} | 82 (6.9) | | 0.004^{†} |
| age, y | | | | | | | | | | |
| Female | 30 (50) | 30 (52) | 1.0 | | 30 (48) | 1.0 | | 60 (50) | 1.0 | |
| Male | 30 (50) | 28 (48) | 0.9 (0.5-1.9) | 0.85 | 32 (52) | 0.9 (0.4-2.0) | 0.86 | 60 (50) | 1.0 (0.5-1.9) | 0.91 |

Smoking Status

| | | | | | | | | | | |
|---|---|---|---|---|---|---|---|---|---|---|
| never | 29 (48) | 31 (52) | 1.0 | | 22 (35) | 1.0 | | 53 (44) | 1.0 | |
| ever | 31 (52) | 27 (47) | 0.8 (0.4-1.7) | 0.54 | 40 (65) | 1.6 (0.8-3.5) | 0.20 | 67 (56) | 1.2 (0.6-2.2) | 0.66 |

Pack Years

| | | | | | | | | | | |
|---|---|---|---|---|---|---|---|---|---|---|
| 0 | 29 (48) | 31 (54) | 1.0 | | 22 (36) | 1.0 | | 53 (45) | 1.0 | |
| 0.1-14.4 | 12 (20) | 11 (19) | 0.8 (0.3-2.2) | | 9 (15) | 0.9 (0.3-2.8) | | 20 (17) | 0.8 (0.3-2.0) | |
| 14.5-33 | 10 (17) | 7 (12) | 0.6 (0.2-1.9) | | 15 (25) | 1.9 (0.7-5.4) | | 22 (19) | 1.3 (0.5-3.1) | |
| 34+ | 9 (15) | 8 (14) | 0.9 (0.3-2.7) | 0.58^{§} | 15 (25) | 2.1 (0.7-6.1) | 0.10^{§} | 23 (19) | 1.3 (0.5-3.4) | 0.48^{§} |

Body Mass Index

| | | | | | | | | | | |
|---|---|---|---|---|---|---|---|---|---|---|
| <25 | 35 (58) | 25 (43) | 1.0 | | 24 (39) | 1.0 | | 49 (40) | 1.0 | |
| 25-29.9 | 14 (23) | 19 (33) | 1.6 (0.7-3.6) | | 25 (40) | 2.3 (1.0-5.4) | | 44 (37) | 1.8 (0.9-3.8) | |
| 30 or greater | 11 (18) | 14 (24) | 1.5 (0.6-3.7) | 0.15^{§} | 13 (21) | 1.1 (0.4-2.8) | 0.17^{§} | 27 (23) | 1.3 (0.6-2.8) | 0.09^{§} |

Genotypes
CFB: rs641153 (R32Q)

| | | | | | | | | | | |
|---|---|---|---|---|---|---|---|---|---|---|
| CC | 46 (80) | 50 (96) | 1.0 | | 47 (92) | 1.0 | | 97 (94) | 1.0 | |
| CT/TT | 11 (19) | 2 (4) | 0.2 (0.03-0.8) | 0.03 | 4 (8) | 0.28 (0.08-1.0) | 0.05 | 6 (6) | 0.23 (0.08-0.70) | 0.01 |

CFH: rs1061170 (Y402H)

| | | | | | | | | | | |
|---|---|---|---|---|---|---|---|---|---|---|
| TT | 28 (50) | 12 (23) | 1.0 | | 8 (16) | 1.0 | | 20 (19) | 1.0 | |
| CT | 18 (32) | 21 (40) | 2.5 (1.0-6.3) | | 22 (43) | 5.4 (1.8-16.0) | | 43 (42) | 3.4 (1.5-7.8) | |
| CC | 10 (18) | 19 (37) | 4.4 (1.6-12.6) | 0.004^{§} | 21 (41) | 9.2 (2.8-30.0) | 0.0002^{§} | 40 (39) | 5.7 (2.3-14.4) | 0.0002^{§} |

CFH: rs1410996

| | | | | | | | | | | |
|---|---|---|---|---|---|---|---|---|---|---|
| TT | 6 (11) | 3 (6) | 1.0 | | 2 (4) | 1.0 | | 5 (5) | 1.0 | |
| CT | 32 (58) | 12 (23) | 0.9 (0.2-4.2) | | 11 (23) | 1.1 (0.2-7.3) | | 23 (23) | 0.8 (0.2-3.1) | |
| CC | 17 (31) | 37 (71) | 4.5 (1.0-21) | 0.001^{§} | 34 (72) | 8.0 (1.3-50) | 0.0002^{§} | 71 (72) | 4.9 (1.3-18.1) | <0.0001^{§} |

C2: rs9332739 (E318D)

| | | | | | | | | | | |
|---|---|---|---|---|---|---|---|---|---|---|
| GG | 48 (84) | 52 (100) | 1.0 | | 44 (88) | 1.0 | | 96 (94) | 1.0 | |
| CG/CC | 9 (16) | 0 (0) | 0^{∥} | 0.04^{#} | 6 (12) | 0.6 (0.2-1.8) | 0.32 | 6 (6) | 0.3 (0.1-0.80) | 0.02 |

LOC387715: rs10490924(A69S)

| | | | | | | | | | | |
|---|---|---|---|---|---|---|---|---|---|---|
| GG | 33 (58) | 20 (38) | 1.0 | | 15 (31) | 1.0 | | 35 (35) | 1.0 | |
| GT/TT | 24 (42) | 33 (62) | 2.2 (1.0-4.7) | 0.05 | 33 (69) | 3.7 (1.6-8.9) | 0.003 | 66 (65) | 2.8 (1.4-5.6) | 0.004 |

C3: rs2230199 (R102H)

| | | | | | | | | | | |
|---|---|---|---|---|---|---|---|---|---|---|
| CC | 36 (62) | 21 (42) | 1.0 | | 23 (49) | 1.0 | | 44 (45) | 1.0 | |
| CG | 19 (33) | 22 (44) | 2.4 (1.0-5.6) | | 19 (40) | 1.7 (0.7-4.1) | | 41 (42) | 2.1 (1.0-4.3) | |
| GG | 3 (5) | 7 (14) | 4.2 (0.9-19.0) | 0.02^{§} | 5 (11) | 2.6 (0.5-12.9) | 0.13^{§} | 12 (12) | 3.4 (0.8-13.4) | 0.02^{§} |

CFI: rs10033900

| | | | | | | | | | | |
|---|---|---|---|---|---|---|---|---|---|---|
| CC | 20 (36) | 19 (36) | 1.0 | | 10 (20) | 1.0 | | 29 (28) | 1.0 | |
| CT | 28 (51) | 20 (38) | 0.8 (0.3-2.0) | | 30 (61) | 2.5 (0.9-6.6) | | 50 (49) | 1.4 (0.6-3.0) | |
| TT | 7 (13) | 14 (26) | 2.3 (0.7-7.3) | 0.23^{§} | 9 (18) | 2.7 (0.7-9.9) | 0.09^{§} | 23 (23) | 2.6 (0.9-7.3) | 0.08^{§} |

Frequency counts for some genotypes and pack years may not add up to the total sample size

Table 1 shows associations between increasing age and all AMD groups. The NV only and total AMD groups were more likely to be smokers or heavy smokers and had higher BMI compared to controls, although these associations are not statistically significant. All genotypes are associated with both types of advanced AMD. There is a significant protective effect of genotype CFB (CT/TT) for GA and a trend for reduced risk of NV, compared with CC genotype. There are strong positive associations between CFH Y402H and CFHrs1410996 CC genotypes (vs TT) and both forms of advanced AMD, and a protective association of C2 CG/C (vs GG) with overall AMD and GA. LOC387715/ARMS2 GT/TT is associated with AMD compared with GG. The C3 GG genotype is positively associated with GA and a similar trend was seen for NV. For CFI, ORs are in the direction of increased risk for the T allele for both GA and NV.

TABLE 2
Levels of Plasma Complement Components/Fragments According to Maculopathy Group

| | | Maculopathy Group | | | | | |
|---|---|---|---|---|---|---|---|
| | Controls | GA | | NV | | Total AMD* | |
| Complement | (n = 60) | (n = 58) | | (n = 62) | | ( n = 120) | |
| Components/ | Median (10th, | Median (10th, | | Median (10th, | | Median (10th, | |
| Fragments | 90th percentile) | 90th percentile) | P-value^{†} | 90th percentile) | P-value^{†} | 90th percentile) | P-value^{†} |
| Bb (μg/mL) | 0.83 (0.46-1.35) | 0.95 (0.60-1.5) | 0.03 | 0.84 (0.53-1.4) | 0.3 | 0.93 (0.56-1.44) | 0.06 |
| C3 (mg/dL) | 110 (86-155) | 113 (82-154) | 0.89 | 115 (86-155) | 0.85 | 114 (83-155) | 0.97 |
| C3a(ng/mL) | 1498 (768-2154) | 1567 (959-2899) | 0.03 | 1647 (704-2613) | 0.22 | 1593.5 (757.5-2738.5) | 0.05 |
| C5a (ng/mL) | 13.5 (7.9-19.9) | 17 (9.3-21.2) | 0.02 | 16 (8.6-24) | 0.09 | 16.2 (9.10-22.9) | 0.02 |
| CFB (μg/mL) | 228 (183-311) | 249 (182-352) | 0.21 | 251 (189-341) | 0.19 | 251 (185-352) | 0.14 |
| iC3b (ng/mL) | 10.02 (4.69-22.72) | 11 (5-29) | 0.42 | 10 (6-25) | 0.97 | 10.37 (5.24-26.15) | 0.63 |
| SC5b-9 (ng/mL) | 403 (176-624) | 332 (164-716) | 0.5 | 335 (188-615) | 0.26 | 333 (184-652) | 0.29 |
| CFI (μg/mL) | 39.9 (27.2-64.1) | 40 (28-56) | 0.66 | 43 (22-57) | 0.97 | 41.5 (22.5-56.4) | 0.82 |
| CFH (μg/mL) | 311.5 (251.5-420) | 289 (231-417) | 0.008 | 295 (246-389) | 0.06 | 293.5 (238-391.5) | 0.009 |
| Factor D (μg/mL) | 3.2 (2.3-4.6) | 3.4 (2.6-4.7) | 0.42 | 3.5 (2.6-4.6) | 0.25 | 3.4 (2.6-4.6) | 0.25 |

*AMD patients who had Geographic Atrophy (GA) or Neovascular (NV) disease in at least one eye.
^{†}P values calculated using Wilcoxon Rank Sum. All p-values reflect comparison between maculopathy group and controls.

Table 2 shows that median levels of activation fragments, Bb, C3a, and C5a, are significantly higher in the
GA group compared with controls. CFH component is significantly lower in the GA group compared with controls
(median of 289 μg/mL vs 312 μg/mL, p=0.008). Results for the total AMD group were similar to the GA group.

TABLE 3
Association Between Plasma Complement
Components/Fragments and AMD, With and
Without Adjustment for Genotypes

| | Model A | | Model B | |
|---|---|---|---|---|
| Plasma | Without genotypes* | | With genotypes^{†} | |
| Complement | OR (95% CI)^{‡} | | OR (95% CI)^{‡} | |
| Component/ | Quartile 4 vs | p | Quartile 4 vs | p |
| Fragment | Quartile 1 | (trend) | Quartile 1 | (trend) |
| Bb | 3.3 (1.3-8.6) | 0.01 | 5.4 (1.3-22.9) | 0.02 |
| C3 | 0.8 (0.3-2.1) | 0.65 | 1.5 (0.4-6.2) | 0.70 |
| C3a | 2.1 (0.8-5.6) | 0.28 | 2.2 (0.5-9.4) | 0.75 |
| C5a | 3.6 (1.2-10.3) | 0.01 | 25.2 (3.7-171.7) | 0.0003 |
| CFB | 1.6 (0.6-4.2) | 0.18 | 3.3 (0.8-12.7) | 0.08 |
| iC3b | 0.8 (0.3-2.1) | 0.79 | 0.7 (0.2-2.7) | 0.81 |
| SC5b-9 | 0.7 (0.3-1.8) | 0.12 | 0.8 (0.2-2.6) | 0.29 |
| CFI | 0.9 (0.3-2.3) | 0.90 | 0.9 (0.2-3.4) | 0.92 |
| CFH | 0.3 (0.09-0.75) | 0.01 | 0.6 (0.1-2.6) | 0.50 |
| Factor D | 1.4 (0.5-3.7) | 0.63 | 3.1 (0.7-12.6) | 0.21 |

*Adjusted for age (60-79, 80+), gender, smoking (ever smoked vs never smoked), body mass index (<25, 25-29.9, >29.9)
^{†}Adjusted for all variables in A plus CFB (CC, CT/TT), CFH Y402H (TT, CT, CC), CFHrs1410966 (TT, CT, CC), C2 (GG, CG/CC), LOC387715 (GG, GT, TT), C3 (CC, CG, GG), CFI (CC, CT, TT).
^{‡}Odds ratios (OR) reflect a comparison between cases and controls in the 4th quartile vs 1st quartile of plasma complement factor.

Table 3 shows a positive association between activation fragment Bb and the total AMD group, both with and without adjustments for genotype (OR=3.3, 95% CI 1.3-8.6; OR=5.4, 95% CI 1.3-22.9, respectively). Similar results were found for C5a (OR=3.6, 95% CI 1.2-10.3; OR=25.2, 95% CI 3.7-171.7), with and without adjusting for genotypes, respectively. CFH component has a significant inverse association with AMD in the model that did not control for genotype (OR=0.3, 95% CI 0.09-0.75, p=0.01), which became non-significant after controlling for genotype.

TABLE 4
Evaluation of Change in Genotype - AMD Associations with Addition of Plasma Complement Components/Fragments*^{†}

| | No Plasma | | Plasma Complement Components/Fragments‡ | | | | | |
|---|---|---|---|---|---|---|---|---|
| | Complement Factors | | Bb | | C5a | | CFH | |
| Genotype | OR (95% CI) | p (trend) | OR (95% CI) | p (trend) | OR (95% CI) | p (trend) | OR (95% CI) | p (trend) |

CFB: rs641153 (R32Q)

| | | | | | | | | |
|---|---|---|---|---|---|---|---|---|
| CC | 1.0 | 0.02 | 1.0 | 0.03 | 1.0 | 0.01 | 1.0 | 0.02 |
| CT/TT | 0.26 (0.08-0.80) | | 0.28 (0.09-0.91) | | 0.24 (0.08-0.74) | | 0.25( 0.08-0.81) | |

CFH: rs1061170 (Y402H)

| | | | | | | | | |
|---|---|---|---|---|---|---|---|---|
| TT | 1.0 | 0.0002 | 1.0 | 0.0002 | 1.0 | <0.0001 | 1.0 | 0.0002 |
| CT | 3.7 (1.6-8.7) | | 4.2 (1.8-10.0) | | 4.3 (1.7-10.6) | | 3.6 (1.5-8.4) | |
| CC | 6.0 (2.3-15.8) | | 5.9 (2.2-15.7) | | 7.9 (2.8-22.4) | | 6.6 (2.4-18.0) | |

CFH: rs1410996

| | | | | | | | | |
|---|---|---|---|---|---|---|---|---|
| TT | 1.0 | <0.0001 | 1.0 | 0.0002 | 1.0 | <0.0001 | 1.0 | <0.0001 |
| CT | 0.9 (0.2-3.7) | | 0.7 (0.2-3.1) | | 1.0 (0.24-4.6) | | 1.0 (0.2-3.6) | |
| CC | 5.2 (1.2-21.4) | | 4.2 (1.0-18.0) | | 7.4 (1.6-33.9) | | 4.9 (1-2-20.4) | |

C2: rs9332739 (E318D)

| | | | | | | | | |
|---|---|---|---|---|---|---|---|---|
| GG | 1.0 | 0.008 | 1.0 | 0.02 | 1.0 | 0.007 | 1.0 | 0.01 |
| CG/CC | 0.20 (0.06-0.67) | | 0.23 (0.07-0.78) | | 0.19 (0.06-0.63) | | 0.22 (0.07-0.74) | |

LOC387715: rs10490924(A69S)

| | | | | | | | | |
|---|---|---|---|---|---|---|---|---|
| GG | 1.0 | 0.007 | 1.0 | 0.01 | 1.0 | 0.001 | 1.0 | 0.009 |
| GT/TT | 2.6 (1.3-5.3) | | 2.5 (1.2-5.2) | | 3.6 (1.7-7.7) | | 2.6 (1.3-5.4) | |

C3: rs2230199 (R102H)

| | | | | | | | | |
|---|---|---|---|---|---|---|---|---|
| CC | 1.0 | 0.01 | 1.0 | 0.02 | 1.0 | 0.03 | 1.0 | 0.008 |
| CG | 2.3 (1.1-4.9) | | 2.1 (0.96-4.6) | | 2.0 (0.90-4.4) | | 2.2 (1.0-4.8) | |
| GG | 3.8 (0.9-15.8) | | 3.1 (0.86-14.6) | | 3.5 (0.83-14.7) | | 5.2 (1.2-23.2) | |

CFI: rs10033900

| | | | | | | | | |
|---|---|---|---|---|---|---|---|---|
| CC | 1.0 | 0.07 | 1.0 | 0.10 | 1.0 | 0.02 | 1.0 | 0.15 |
| CT | 1.6 (0.71-3.4) | | 1.6 (0.71-3.5) | | 1.8 (0.78-4.0) | | 1.5 (0.68-3.4) | |
| TT | 2.6 (0.90-7.7) | | 2.5 (0.82-7.4) | | 3.9 (1.2-12.2) | | 2.1 (0.72-6.4) | |

*Adjusted for age (60-79, 80+), gender, smoking (ever smoked vs never smoked), body mass index (<25, 25-29.9, >29.9)
^{†}Odds Ratios (OR) reflect a comparison of all cases vs controls.
^{‡}Quartiles

Table 4 shows the change in the association between genotype and AMD, with addition of complement factors Bb, C5a, and CFH to the models. The ORs for risk of AMD increase for both CFH loci, LOC, and CFI genotypes, with the addition of activation fragment C5a into the statistical models. There is also an increase in the OR for the C3 gene when CFH component was added to the model. The associations of the other genetic loci with AMD were not materially changed after inclusion of complement components and activation fragments one at a time.

TABLE 5
Associations Between Plasma Complement Components/Fragments, Smoking, and Body Mass Index

| | Smoking (Ever vs. Never)† | | Smoking (Ever vs. Never)‡ | | | BMI^{§} | | BMI^{∥} | | |
|---|---|---|---|---|---|---|---|---|---|---|
| Complement | Controls (N = 60) | | All Subjects (N = 180) | | | Controls (N = 60) | | All Subjects (N = 180) | | |
| Components/ | | (p | | (p | p inter- | | (p | | (p | p inter- |
| Fragments* | β ± SE^{#} | value)** | β ± SE^{#} | value)** | action | β ± SE^{#} | value)** | β ± SE^{#} | value)** | action |
| Bb | 0.05 ± 0.11 | (0.65) | 0.02 ± 0.10 | (0.15) | 0.46 | −0.02 ± 0.11 | (0.83) | −0.11 ± 0.06 | (0.08) | 0.45 |
| C3 | −0.03 ± 0.06 | (0.59) | 0.05 ± 0.04 | (0.18) | 0.23 | 0.09 ± 0.06 | (0.17) | 0.10 ± 0.03 | (0.005) | 0.93 |
| C3a | 0.001 ± 0.10 | (0.99) | 0.06 ± 0.07 | (0.40) | 0.75 | 0.25 ± 0.09 | (0.01) | 0.09 ± 0.07 | (0.17) | 0.07 |
| C5a | −0.03 ± 0.09 | (0.76) | 0.02 ± 0.06 | (0.77) | 0.54 | 0.04 ± 0.09 | (0.69) | −0.03 ± 0.06 | (0.62) | 0.46 |
| CFB | 0.05 ± 0.07 | (0.51) | −0.006 ± 0.04 | (0.88) | 0.17 | 0.13 ± 0.07 | (0.06) | 0.12 ± 0.04 | (0.001) | 0.54 |
| iC3b | −0.13 ± 0.16 | (0.41) | −0.12 ± 0.09 | (0.17) | 0.96 | 0.31 ± 0.16 | (0.05) | 0.10 ± 0.09 | (0.26) | 0.11 |
| SC5b-9 | −0.15 ± 0.13 | (0.24) | −0.12 ± 0.07 | (0.17) | 0.55 | 0.11 ± 0.13 | (0.39) | −0.05 ± 0.08 | (0.56) | 0.41 |
| CFI | −0.03 ± 0.09 | (0.73) | −0.005 ± 0.05 | (0.92) | 0.70 | 0.01 ± 0.09 | (0.89) | 0.04 ± 0.05 | (0.47) | 0.82 |
| CFH | −0.07 ± 0.05 | (0.18) | −0.02 ± 0.03 | (0.60) | 0.28 | 0.12 ± 0.05 | (0.03) | 0.12 ± 0.03 | (0.0006) | 0.95 |
| Factor D | 0.09 ± 0.08 | (0.25) | 0.06 ± 0.04 | (0.13) | 0.50 | 0.08 ± 0.08 | (0.34) | 0.03 ± 0.04 | (0.44) | 0.38 |

p interaction is the interaction between smoking or BMI and AMD status
*Log values
^{†}Controlling for age, gender.
^{‡}Controlling for age, gender, and AMD status (total AMD, controls).
^{§}Body Mass Index (BMI) categories: <25, 25+. Controlling for age and gender.
^{∥}BMI categories: <25, 25+. Controlling for age, gender, and AMD status (total AMD, controls).
^{#}Beta ± Standard Error
**P values for controls only were calculated by multiple regression of log complement component/fragment on age 80+ vs. <80 and gender. P values for all subjects were caculated the same as controls, and in addition controlled for AMD status.

Significant associations were found between BMI ≧25 and increased levels of fragments C3a and iC3b, and component CFH among controls (Table 5). When the sample size was increased to include the entire study population to further assess these relationships and controlled for AMD status, CFH remained significant, CFB and C3 became significant (p=0.001 and 0.005, respectively), whereas C3a and iC3b became non-significant. No significant association was found between complement components or fragments and smoking.

Interactions between genotypes and components and fragments were also assessed. A significant interaction was found between component CFH and the protective CFB genotype (p=teraction=0.04). This indicates that the component CFH is inversely associated with risk of AMD among individuals with the CFB genotype CC, but is unrelated to risk of AMD in the presence of a protective allele CFB genotype of CT/TT.

TABLE 6
Associations Between Complement Components/Fragments and Genotype Among All Subjects

| | Complement Component/Fragment* | | | | | | | |
|---|---|---|---|---|---|---|---|---|
| | Bb | | C3a | | C5a | | CFH | |
| Genotype | LS Means ± SE^{†} | (p value) | LS Means ± SE^{†} | (p value) | LS Means ± SE^{†} | (p value) | LS Means ± SE^{†} | (p value) |

CFB: rs641153 (R32Q)

| | | | | | | | | |
|---|---|---|---|---|---|---|---|---|
| CC | −0.28 ± 0.08 | 0.57 | 7.41 ± 0.09 | 0.26 | 2.79 ± 0.08 | 0.96 | 5.79 ± 0.06 | 0.80 |
| CT/TT | −0.33 ± 0.12 | | 7.20 ± 0.14 | | 2.78 ± 0.12 | | 5.80 ± 0.07 | |

CFH: rs1061170 (Y402H)

| | | | | | | | | |
|---|---|---|---|---|---|---|---|---|
| TT | −0.23 ± 0.10 | 0.47^{‡} | 7.34 ± 0.12 | 0.43^{‡} | 2.83 ± 0.10 | 0.51^{‡} | 5.78 ± 0.06 | 0.52^{‡} |
| CT | −0.40 ± 0.11 | | 7.32 ± 0.12 | | 2.77 ± 0.10 | | 5.78 ± 0.06 | |
| CC | −0.29 ± 0.11 | | 7.24 ± 0.13 | | 2.75 ± 0.11 | | 5.82 ± 0.06 | |

CFH: rs1410996

| | | | | | | | | |
|---|---|---|---|---|---|---|---|---|
| TT | −0.51 ± 0.15 | 0.16^{‡} | 7.40 ± 0.17 | 0.92^{‡} | 2.86 ± 0.14 | 0.45^{‡} | 5.76 ± 0.08 | 0.77^{‡} |
| CT | −0.22 ± 0.10 | | 7.22 ± 0.11 | | 2.76 ± 0.10 | | 5.81 ± 0.06 | |
| CC | −0.19 ± 0.10 | | 7.29 ± 0.11 | | 2.73 ± 0.10 | | 5.81 ± 0.05 | |

C2: rs9332739 (E318D)

| | | | | | | | | |
|---|---|---|---|---|---|---|---|---|
| GG | −0.20 ± 0.07 | 0.13 | 7.22 ± 0.08 | 0.21 | 2.70 ± 0.07 | 0.16 | 5.74 ± 0.04 | 0.18 |
| CG/CC | −0.42 ± 0.14 | | 7.39 ± 0.16 | | 2.87 ± 0.14 | | 5.85 ± 0.08 | |

LOC387715: rs10490924(A69S)

| | | | | | | | | |
|---|---|---|---|---|---|---|---|---|
| GG | −0.35 ± 0.10 | 0.40 | 7.33 ± 0.11 | 0.49 | 2.87 ± 0.09 | 0.02 | 5.77 ± 0.05 | 0.30 |
| GT/TT | −0.27 ± 0.09 | | 7.27 ± 0.11 | | 2.70 ± 0.09 | | 5.81 ± 0.05 | |

C3: rs2230199 (R102H)

| | | | | | | | | |
|---|---|---|---|---|---|---|---|---|
| CC | −0.33 ± 0.09 | 0.58^{‡} | 7.20 ± 0.10 | 0.07^{‡} | 2.69 ± 0.09 | 0.04^{‡} | 5.75 ± 0.05 | 0.15^{‡} |
| CG | −0.26 ± 0.09 | | 7.47 ± 0.10 | | 2.82 ± 0.09 | | 5.71 ± 0.05 | |
| GG | −0.34 ± 0.14 | | 7.24 ± 0.16 | | 2.84 ± 0.13 | | 5.91 ± 0.08 | |

CFI: rs10033900

| | | | | | | | | |
|---|---|---|---|---|---|---|---|---|
| CC | −0.32 ± 0.10 | 0.74^{‡} | 7.39 ± 0.12 | 0.07^{‡} | 2.82 ± 0.10 | 0.29^{‡} | 5.80 ± 0.06 | 0.65^{‡} |
| CT | −0.30 ± 0.09 | | 7.30 ± 0.10 | | 2.82 ± 0.09 | | 5.80 ± 0.05 | |
| TT | −0.30 ± 0.11 | | 7.22 ± 0.13 | | 2.72 ± 0.11 | | 5.77 ± 0.06 | |

*Log values
^{†}Least square means ± standard error, controlling for age, gender, AMD status, CFB (CC, CT/TT), CFH Y402H (TT, CT, CC), CFHrs1410966 (TT, CT, CC), C2 (GG, CG/CC), LOC387715 (GG, GT, TT), C3 (CC, CG, GG), CFI (CC, CT, TT).
^{‡}P value for trend.

Controlling for all genotypes, significant associations were found between fragment C5a and the LOC387715/ARMS2 and C3 genotypes although the differences were small (Table 6). For the LOC387715/ARMS2 genotype the least square (LS) mean for C5a was lower with the addition of the risk allele T. For the C3 genotype the trend for increasing C5a is significant with the addition of each risk allele. Bb LS means are negative because they are on a log scale and have values less than one in some instances.

TABLE 7
C Statistics for Age-Related Macular Degeneration Based on
Models with Demographic, Behavioral, Genetic Factors, and
C5a, Bb, and C3a

| Model | Variables | C-statistic |
|---|---|---|
| 1 | Age, gender, smoking, BMI | 0.612 ± 0.050 |
| 2 | Age, gender, smoking, BMI, | 0.832 ± 0.036 |
| | CFB: rs641153 (R32Q), CFH: | |
| | rs1061170 (Y402H), CFH: | |
| | rs1410996, C2: rs9332739 | |
| | (E318D), LOC387715: rs10490924 | |
| | (A69S), C3: rs2230199 (R102H), | |
| | CFI: rs10033900 | |
| 3 | Age, gender, smoking, BMI, | 0.844 ± 0.035 |
| | CFB: rs641153 (R32Q), CFH: | |
| | rs1061170 (Y402H), CFH: | |
| | rs1410996, C2: rs9332739 | |
| | (E318D), LOC387715: rs10490924 | |
| | (A69S), C3: rs2230199 (R102H), | |
| | CFI: rs10033900, C3a fragment | |
| 4 | Age, gender, smoking, BMI, | |
| | CFB: rs641153 (R32Q), CFH: | 0.870 ± 0.031 |
| | rs1061170 (Y402H), CFH: | |
| | rs1410996, C2: rs9332739 | |
| | (E318D), LOC387715: rs10490924 | |
| | (A69S), C3: rs2230199 (R102H), | |
| | CFI: rs10033900, Bb fragment | |
| 5 | Age, gender, smoking, BMI, | 0.895 ± 0.028 |
| | CFB: rs641153 (R32Q), CFH: | |
| | rs1061170 (Y402H), CFH: | |
| | rs1410996, C2: rs9332739 | |
| | (E318D), LOC387715: rs10490924 | |
| | (A69S), C3: rs2230199 (R102H), | |
| | CFI: rs10033900, C5a fragment | |
| 6 | Age, gender, smoking, BMI, | 0.944 ± 0.020 |
| | CFB: rs641153 (R32Q), CFH: | |
| | rs1061170 (Y402H), CFH: | |
| | rs1410996, C2: rs9332739 | |
| | (E318D), LOC387715: rs10490924 | |
| | (A69S), C3: rs2230199 (R102H), | |
| | CFI: rs10033900, C3a, Bb, C5a | |
| | fragments | |

* p value (model 1 vs 2, p = 0.001; 2 vs 3 = 0.63; 2 vs 4 = 0.043; 2 vs 5 = 0.029; 2 vs 6 = <0.001)

C statistics for all cases versus controls were calculated for various models to assess the predictability of advanced AMD (Table 6): Model 1 (age, gender, AMD status, smoking, BMI); Model 2 (all variables in Model 1 plus genetic variants CFB, CFHY402H, CFH:rs1410996, C2, LOC387715/ARMS2, C3, and CFI; and Models 3, 4, 5, 6 (Model 2 plus fragments C3a, Bb, and C5a, and all three markers, respectively). There were significant increases in C statistics upon adding Bb (0.870) and C5a (0.895) to Model 2 (p=0.029, 0.043, respectively). Combining C3a, Bb, and C5a into the model resulted in a C statistic of 0.944, and a p value of <0.001 compared to Model 2. The frequency distribution of risk scores was plotted separately for cases and controls for Model 6, selecting a cutoff of 4 (risk score ≧0), yields a sensitivity of 88% and a specificity of 73% (FIGURE). In general, cases had higher risk scores than controls.

### REFERENCE

1. Gold B, Merriam J E, Zernant J et al. Nature Genetics. 2006; 38:458-462.
2. Maller, J. et al. Nature Genetics. 2006; 38:1055-1059.
3. Hageman G S, Anderson D H, Johnson L V et al. Proc Natl Acad Sci USA. 2005; 102:7227-7232
4. Klein R J, Zeiss C, Chew E Y, et al. Science. 2005; 308:385-389
5. Edwards A O, Ritter R, Abel K J et al. Science. 2008; 308:421-424.
6. Li M, Atmaca-Sonmez P, Othman M, et al. Nature Genetics. 2006; 38:1049-1054.
7. Haines J L, Hauser M A, Schmidt S et al. Science. 2005; 308: 419-421.
8. Mailer, J. Et al. Nature Genetics. 2007; 39: 1200-1201.
9. Fagerness, J A et al. European Journal of Human Genetics. 2009 17(1): 100-104.
10. Jakobsdottir J, Conley Y, Weeks D E, et al. Am J Hum Gen. 2005 77 (3):389-407.
11. Dewan A., Liu M, Hartman S. et al. Science. 2006; 314:989-992.
12. Yang Z, Camp N J, Sun H, et al. Science. 2006; 314:992-993.
13. Kanda A, Chen W, Othman M, et al. Proc Natl Acad Sci USA. 2007; 104:16227-16232.
14.Seddon, J. M., Cote, J., Davis, N. & Rosner, B. Arch Ophthalmol. 2003; 121: 785-92.
15. Seddon J M, Gensler G, Milton R C, Klein M L, Rifai N. JAMA. 2004; 291 (6):704-710
16. Seddon J M, Gensler G, Klein M L, Milton R C. Nutrition 2006; 22: 441-443.
17. Seddon J M, George S, Rosner B, Klein M. Human Heredity. 2006; 61:157-165.
18. Seddon J M, Reynolds R, Maller J, Fagerness J A, Daly M J, Rosner B. IOVS 2009; 50: 2044-2053.
19. Snow, K. K., Cote, J., Yang, W., Davis, N. J. & Seddon, J. M. Am J Ophthalmol. 2002; 134:842-8.
20.Seddon, J. M., Santangelo, S. L., Book, K., Chong, S. & Cote, J. A. Am J Hum Genet. 2003; 73:780-90.
21. Seddon, J. M., Cote, J., Page, W. F., Aggen, S. H. & Neale, M. C. Arch Ophthalmol 2005; 123: 321-7.
22. Seddon J M, Sharma S, Adelman R. Ophthalmology. 2006; 113: 260-266.
23. Harbeck R J and P C Giclas. Diagnostic Immunology Laboratory Manual, Raven Press, NY, 1991.
24. Seddon J M, Francis P J, George S, Schultz D, Rosner B, Klein M. JAMA. 2007; 297:1793-1800.
25. Hageman G S, Luthert P J, Victor Chong N H, Johnson L V, Anderson D H, Mullins R F. Progress in Retinal and Eye Research. 2001; 20(6):705-732.
26. Zhou J, Jang Y P, Kim S R, Sparrow J R. Proc Nati Acad Sci USA. 2006; 103(44):16182-16187.
27. Atkinson, J P. Complement System. Kelley's Textbook of Rheumatology Eighth Edition. Firestein G S, Budd R C, Harris E D, McInnes I B, Ruddy S, Sergent J S . Saunders Elsevier. 323-337, 2009.
28. Bora N S, Sankaranaray K et al. The Journal of Immunology. 2006; 177:1872-1878.
29. Bora P, Sohn J H et al. The Journal of Immunology. 2005;174: 491-497.
30.Nozaki, Raisler et al. Proc Natl Acad Sci USA. 2006; 103 (7):2328-2333.
31. Huber-Lang Met al. Nat Med. 2006 June; 12(6):682-7.
32. Scholl H P N, Issa P C, Walier M, Janzer S et al. PLOSOne. 2008;3(7).
33. Pattrick M, Luckett J, Yue L, Stover C. Mol Immunol. 2009; 46:755-760.
34. Sivaprasad et al. Arch Ophthalmol. 2007; 125:515-519
35. R T White, D Damm, N Hancock, B S Rosen, B B Lowell, P Usher, J S Flier and B M Spiegelman J. Biol. Chem., Vol. 267, Issue 13, 9210-9213, 05, 1992.
36. Paglalunga S. et al. Am J Endocrinol Metab. 2008; 294:E521-E529.
37. Wamba P C F et al. Eur J Endocrinol. 2008; 159: 781-790.
38. Hernandez-Mijares A, Jarabo-Bueno M M, Lopez-Ruiz A et al. Int J Obes (Lond.). 2007; 31(6):927-32.
39. Warnberg J, Nova E, Moreno L A, Romeo J et al. Am J Clin Nutr. 2006; 84 (3):505-512.

What is claimed is:

1. A method for determining AMD risk in a patient, comprising: obtaining a patient blood sample and determining the serum or blood plasma levels of complement factor polypeptides, wherein elevated serum or plasma levels of one or more complement factor polypeptides are indicative of susceptibility for or an increased risk of developing AMD, or an increased risk of progression of AMD in the patient.

2. The method of claim 1, wherein the complement factor polypeptides are C3, CFB, Factor I, CFH, Factor D, Bb, C3a, iC3b, C5a, SC5b-9 and related complement pathway polypeptides.

3. The method of claim 2, wherein elevated serum or plasma levels of complement factor polypeptides is determined using an antibody to the complement factor polypeptides.

4. The method of claim 2, wherein elevated serum or plasma levels of complement factor polypeptides is determined using a radial immunodiffusion assay or an ELISA, and nephelometric methods.

5. A kit for determining AMD risk in a patient, comprising: an immunoassay having antibodies directed to one or more complement factor polypeptides, reference standards comprising physiological ranges of one or more of the complement factor polypeptides, suitable packaging and instructions for use.

6. The kit of claim 5, wherein the complement factor polypeptides are C3, CFB, Factor I, CFH, Factor D, Bb, C3a, iC3b, C5a, SC5b-9 and related complement pathway polypeptides.

7. A diagnostic system comprising: an array, the array having reference locations and diagnostic locations, the reference locations having a known quantity of an antibody to a complement factor polypeptide at each location with the known quantity of antibody differing in quantity at each location, and the diagnostic locations having a known quantity of an antibody to a complement factor polypeptide at each location with the known quantity of antibody common to each location, the diagnostic system further comprising reference standards of one or more complement factor polypeptides, an array reader, an image processor, a database having data records and information records, a processor, and an information output; wherein the system compiles and processes patient data relative to the serum or plasma levels of complement factors in a patient, and where the system outputs information relating to the statistical probability of the patient having susceptibility for or an increased risk of developing AMD, or an increased risk of progression of AMD in the patient, based on the serum or plasma levels of complement factor polypeptides in the patient.

8. The system of claim 7 wherein the complement factor polypeptides are C3, CFB, Factor I, CFH, Factor D, Bb, C3a, iC3b, C5a, SC5b-9 and related complement pathway polypeptides.

9. A method of using the system of claim 7, comprising obtaining a patient blood sample and determining the serum or plasma levels of complement factor polypeptides in the blood sample, wherein elevated serum or plasma levels of one or more complement factor polypeptides indicate a susceptibility for or an increased risk of developing AMD, or an increased risk of progression of AMD in the patient.

10. The method of claim 1 further comprising, determining the presence or absence of a particular allele at a polymorphic site associated with one or more complement pathway genes, wherein the allele indicates a susceptibility to AMD, a protective phenotype for AMD, or a neutral genotype for AMD, thereby indicating AMD risk in the patient.

11. The method of claim 10, wherein the allele at a polymorphic site is a single nucleotide polymorphism associated with one or more complement pathway genes including rs1061170 (Factor H gene), rs1410996 (Factor H gene), rs9332739 (Complement Factor 2 gene), rs641153 (Factor B gene), rs2230199 (C3 gene); and rs10033900 (Complement Factor I), and other genes such as rs10490924 (at LOC387715/ARM5 on chromosome 10 region).

12. The method of claim 10, wherein the presence or absence of a particular allele is detected by a hybridization.

13. The system of claim 7, further comprising an array of genes encoding one or more complement pathway proteins.

14. The system of claim 13, wherein the genes include single nucleotide polymorphism associated with one or more complement pathway genes including rs1061170 (Factor H gene), rs1410996 (Factor H gene), rs9332739 (Complement Factor 2 gene), rs641153 (Factor B gene), rs2230199 (C3 gene); and rs10033900 (Complement Factor I) and other genes such as rs10490924 (at LOC387715/ARM5 on chromosome 10 region).

15. A method of using the diagnostic system of claim 14, comprising contacting a subject sample to the diagnostic array under high stringency hybridization conditions; inputting patient information into the system; and obtaining from the system information relating to the statistical probability of the patient developing AMD.

16. A method of making the diagnostic array of claim 10, comprising: applying to a substrate at a plurality particular address on the substrate a sample of the individual purified polynucleotide compositions comprising rs1061170 (Factor H gene), rs1410996 (Factor H gene), rs9332739 (Complement Factor 2 gene), rs641153 (Factor B gene), rs2230199 (C3 gene); and rs10033900 (Complement Factor I) and other genes such as rs10490924 (at LOC387715/ARM5 on chromosome 10 region).

17. A method for diagnosing AMD or a susceptibility to AMD in a subject comprising evaluating plasma levels of one or more of the complement pathway factor polypeptides of claim 2 and one or more of the gene polymorphisms of claim 11, and correlating the plasma levels of the polypeptides and the presence or absence of the gene polymorphisms, with medical, behavioral and environmental risk factors, thereby determining the patient's risk for AMD.

18. The method of claim 14, wherein the risk factors include hyperlipidemia, aberrant cholesterol levels, high blood pressure, obesity, smoking, vitamin and dietary supplement intake, patient use of alcohol or drugs, poor diet and sedentary lifestyle.

19. The method of claim 1, wherein high serum or plasma protein levels of complement factor polypeptides Bb, C3a, C5a and low serum or plasma protein levels of complement Factor H are indicative of susceptibility for or an increased risk of developing AMD, or an increased risk of progression of AMD in the patient.

20. The method of claim 10, wherein the predictive value of complement factors Bb and C5a are positively associated with AMD with or without the adjustments for genotype, and complement Factor H has a reverse correlation with AMD without genotype adjustments but becomes non-significant after genotype adjustments.

21. The method of claim 10, wherein addition of complement factors to the polymorphism-based prediction models for AMD improve the statistical significance of the correlation with AMD.

22. The method of claim 18, wherein susceptibility for or an increased risk of developing AMD, or an increased risk of progression of AMD increases when the positive risk factor of complement factor C5a is integrated with the positive risk factors of genetic polymorphisms in Factor H-, Factor I- and LOC, in associated genotype prediction models.

23. The method of claim 10, wherein the predictive value of complement factors that are markers of chronic complement activation are significantly elevated in AMD patients compared to non-AMD patients.

24. The method of claim 20, wherein the complement factors are Ba, C3d and Factor D.
